(19) United States
(12) Patent Application Publication
(10) Pub. No.: US 2012/0004122 A1
(43) Pub. Date: Jan. 5, 2012

(54) Diagnostic Marker for Migraine and Use Thereof

(76) Inventors: Eiichiro Nagata, Tokyo (JP); Norihiro Suzuki, Tokyo (JP)

(21) Appl. No.: 12/066,900

(22) PCT Filed: Sep. 14, 2006
(86) PCT No.: PCT/JP2006/318244

§ 371 Date: Oct. 8, 2009

(30) Foreign Application Priority Data
Sep. 15, 2005 (JP) 2005-268605

(51) Int. Cl. C40B 30/04; G01N 33/566; C12Q 1/68

(52) U.S. Cl. 506/9; 435/6.12; 435/7.1

(57) ABSTRACT

The present invention is directed to providing diagnostic markers that enable diagnosis of migraine in a simple and convenient manner and to methods for using them. An α-fodrin (spectrin) gene-related substance or an HPCAL1 gene-related substance are used as a diagnostic marker. As a method for assaying the diagnostic marker, peripheral blood lymphocytes obtained from patients with migraine are immortalized, cell extract is prepared, and the expression level of the diagnostic marker is examined. This assay method enables methods for screening for efficacy and methods for screening for a novel therapeutic agent for migraine.

### CROSS REFERENCE TO RELATED APPLICATIONS

This application claims the benefit of priority to Japanese Patent Application No. 2005-268605, filed on Sep. 15, 2005, which is incorporated herein by reference.

### TECHNICAL FIELD

The present invention relates to diagnostic markers for migraine and methods for using the same.

### BACKGROUND ART

Migraine is what is called a functional headache that is not associated with structural defect. The number of patients with migraine is relatively large. However, since it is not a fatal disease and experiments and research using animals are difficult, its pathological condition has yet to be clarified.

Meanwhile, for particular diseases under analysis, gene diagnosis which enables accurate diagnosis by using a gene-related substance as a marker for diagnosis has become possible (refer to, for example, Japanese Laid-Open Application No. 2003-116543 and its Japanese Domestic Republication No. 2003-74736). Diagnosis with markers involves relatively few uncertain factors, such as, for example, difference in symptom depending on the individual, which are involved in symptom-based diagnosis.

### DISCLOSURE OF THE INVENTION

### Problems to be Solved by the Invention

The object of the present invention is to provide a diagnostic marker which enables diagnosis of migraine in a simple and convenient manner and a method for using it.

### Means for Solving the Problems

To solve the above-mentioned problem, the inventors of the present application have cultured lymphocytes harvested from healthy subjects and patients with migraine, and prepared cRNAs using total RNAs of the cells from each group as templates. By using the cRNAs, the mRNA expression level was analyzed on the RNA arrays (included in a kit) on which about 20,000 known human genes are spotted as probes. The probes in the migraine group whose expression levels are more than two times compared with those in the healthy group were identified and analyzed. As a result, they were roughly classified into cytoskeletal genes and calcium-related genes. Then, α-fodrin (also referred to as spectrin), a membrane-lining protein, and HPCAL1 (hippocalcin-like 1), a calcium-binding protein present in the brain, were identified as one of the cytoskeletal genes and one of the calcium-related genes, respectively. The present invention has thus been accomplished.

That is, first, the diagnostic marker according to the present invention is a diagnostic marker for migraine, which contains an α-fodrin gene-related substance or an HPCAL1 (hippocalcin-like 1) gene-related substance. This gene-related substance may be an mRNA, a cDNA, or a protein.

The diagnostic kit according to the present invention is a diagnostic kit for migraine containing the aforementioned diagnostic marker.

Next, the assay method according to the present invention includes the step of examining an expression level of the α-fodrin gene or the HPCAL1 gene in a lymphocyte isolated from a vertebrate individual. The isolated cell may further be immortalized and the immortalization may be performed using EB virus.

Then, the method for screening for a therapeutic agent for migraine according to the present invention includes isolating a lymphocyte from a patient with migraine, administering a therapeutic agent for migraine effective for a patient with migraine to the isolated lymphocyte, examining an expression level of the α-fodrin gene or the HPCAL1 gene in the isolated cell before and after the administration, and selecting a therapeutic agent for migraine which decreases the expression level after the administration compared with that before the administration. This screening method can be performed, by administering to a lymphocyte a substance which serves as a candidate for a novel therapeutic agent for migraine, to select a therapeutic agent for migraine. And, in order to examine the expression level, the amount of mRNA, cDNA, or protein derived form the α-fodrin gene or the HPCAL1 gene may be measured. In addition, the isolated cell may further be immortalized and the immortalization may be performed using EB virus.

Further, the diagnosis method according to the present invention includes examining an expression level of the α-fodrin gene or the HPCAL1 gene in a lymphocyte isolated from a subject. The diagnosis method may include the steps of administering a therapeutic agent for migraine to a lymphocyte isolated from the subject, examining an expression level of the α-fodrin gene or the HPCAL1 gene in the isolated cell before and after the administration, and diagnosing the subject having the expression level that decreased after the administration compared with that before the administration as being afflicted with migraine. The isolated cell may further be immortalized the immortalization may be performed using EB virus.

It should be noted that the term “diagnostic marker” as used herein refers to a relevant gene and its related substance serving as an indicator (i.e. marker) for evaluating the state or action of a particular object having a correlation with the expression level of the gene having the aforementioned state of action. The diagnostic marker encompasses, for example, a gene itself, as well as an mRNA as a transcript, a peptide as a translation product, and a protein as an end product of gene expression, in the process of gene expression, etc. The “expression level of a diagnostic marker” as used herein refers to, when a gene itself is used, the expression level of the relevant gene, and when a gene marker other than a gene is used, it refers to the amount of expression of the gene from which the diagnostic marker is derived at the transcription level (when the marker is a transcript etc.), or at the transcription level (when the marker is a polypeptide or a protein, etc.). In addition, in the case of humans, the above-mentioned α-fodrin gene are the HPCAL1 gene and defined by the accession numbers BC 053521 and BC 017028 in the International Nucleotide Sequence Database Collaboration, respectively. The gene to be used for implementation of the present invention is not limited in terms of the species from which it is derived, but it encompasses all homologs and orthologs.

### BRIEF DESCRIPTION OF THE DRAWINGS

FIG. 1 is a graph showing the ratio of the gene expression level of each of the α-fodrin gene and the HPCAL1 gene in the migraine group to that in the healthy group in an example according to the present invention.

FIG. 2 is a graph showing the relative values of the gene expression levels of the α-fodrin gene in the migraine group and the healthy group before and after the administration of sumatriptan (A), aspirin (B), and Gaster (famotidine) (C) in an example according to the present invention.

FIG. 3 shows the results of examining the expression level of α-fodrin in the pathological animal model for migraine by RT-PCR (A) and real-time PCR (B). S indicates the cerebral hemisphere where CSD has occurred on stimulation; and N indicates the cerebral hemisphere which has not been stimulated.

### BEST MODE FOR CARRYING OUT THE INVENTION

Embodiments of the present invention accomplished based on the above-described findings are hereinafter described in detail by giving Examples. Unless otherwise explained, methods described in standard sets of protocols such as J. Sambrook and E. F. Fritsch & T. Maniatis (Ed.), “Molecular Cloning, a Laboratory Manual (3rd edition), Cold Spring Harbor Press and Cold Spring Harbor, N.Y. (1989); and F. M. Ausubel, R. Brent, R. E. Kingston, D. D. Moore, J. G. Seidman, J. A. Smith, and K. Struhl (Ed.), “Current Protocols in Molecular Biology,” John Wiley & Sons Ltd., and/or their modified/changed methods are used. When using commercial reagent kits and measuring apparatus, unless otherwise explained, protocols attached to them are used.

The object, characteristics, and advantages of the present invention as well as the idea thereof will be apparent to those skilled in the art from the descriptions given herein. It is to be understood that the embodiments and specific examples of the invention described herein below are to be taken as preferred examples of the present invention. These descriptions are only for illustrative and explanatory purposes and are not intended to limit the invention to these embodiments or examples. It is further apparent to those skilled in the art that various changes and modifications may be made based on the descriptions given herein within the intent and scope of the present invention disclosed herein.

### ===Diagnostic Marker===

The diagnostic marker for migraine according to the present invention includes the related substances of the two genes having a specifically high expression level in the peripheral blood lymphocytes of patients with migraine, the α-fodrin (spectrin) gene and the HPCAL1 (hippocalcin-like 1) gene. The gene-related substances include DNAs, RNAs, and proteins.

When the expression level of each of the aforementioned two genes in lymphocytes derived from the healthy group is compared with that derived from the migraine group, both genes exhibit a significantly higher expression level in the migraine group. Further, when the expression levels of these genes are compared between before and after the addition of a therapeutic agent for migraine to immortalized cells, the expression levels decreased after the addition of the therapeutic agent.

It is therefore concluded that the two genes and their related substances are useful, in peripheral blood lymphocytes, as indicators, i.e. markers, for judging the onset of migraine in patients with migraine or candidate patients with migraine.

### ===Assay for Detecting a Diagnostic Marker===

The assay for detecting a diagnostic marker in samples includes the step of examining the expression level of the relevant gene or its related substance.

In the assay according to the present invention, the expression level of the aforementioned diagnostic marker was examined by quantifying the diagnostic marker. Lymphocytes contained in the peripheral blood of the animal to be diagnosed can be used as the samples to be measured. Also in other body tissues, as long as the difference in the expression level of the gene-related substance as mentioned above is confirmed, samples derived from the tissue may be used for the assay, but the method using lymphocytes is preferred in that it is relatively less invasive for the animal to be diagnosed.

Immortalized cells, prepared using cells such as isolated lymphocytes which have been subjected to treatment of immortalization, can also be used as samples for this assay. In this case, since the immortalized cells can be passaged in the same condition for a long time, the above-mentioned measuring method becomes possible to be repeatedly carried out under the same conditions, thereby increasing the reproducibility of this assay. Furthermore, since only one blood drawing is required from the animal to be diagnosed, the number of invasive procedures to animal subjects can be reduced when the present invention is implemented. For treatment for cell immortalization, infection with EB virus may be used. Also, infection with SV40 virus and other general cell immortalization techniques may be used. It can be said that the assay method using such immortalized cells provides a new experimental model for diagnosis and clarification of the cause of migraine.

In order to examine the expression of the above-mentioned diagnostic marker in isolated lymphocytes, first, cell extract may be prepared and then expression of the diagnostic marker in it may be examined.

There are various known methods for detecting the expression. First, when the gene-related substance is an mRNA or a cDNA, the Northern blotting method, dot blotting method, PCR method, etc. can be used. Particularly preferred among these is the use of the quantitative PCR method capable of high-sensitivity measurement in that only a small quantity of samples is required for measurement. More preferred is the use of the real-time PCR method, an improvement over quantitative PCR, in that it enables more accurate measurement.

Primers to be used for the quantitative PCR method are not particularly limited, as long as it can specifically detect the diagnostic marker of interest, but oligonucleotides consisting of 12 to 26 nucleotides are preferred. Their nucleotide sequences are determined based on the sequence information (refer to the sequence listing) of the known genes of the vertebrate or human to be diagnosed. Then, the primers containing the determined sequences can be prepared with, for example, a DNA synthesizer. Furthermore, even when the genes of the animal to be diagnosed are unknown, on the basis of the sequence information of homologs or orthologs of the known related genes, this assay can be implemented by estimating sequences based on assumption of the similarity between animal species and by degenerating primer sequences.

Meanwhile, when the gene-related substance serving as the marker according to the present invention is a translation product (a polypeptide) or an end product (a protein) of the gene, the substance can be assayed by immunochemical approaches using a polyclonal antibody, a monoclonal antibody, etc., specific to the gene marker, such as radioimmunoassay (RIA), enzyme immunoassay (EIA), and other immunochemical assays. Alternatively, when the method for purifying the polypeptide or protein to serve as the marker is known, after the purification has been performed, a standard polypeptide protein assay method, for example, the Lowry method etc., can be carried out besides the aforementioned immunochemical assays. As the antibodies to be used for measurement, commercially available antibodies whose specificities have been confirmed may be used, or, alternatively, new antibodies may be prepared by the standard antibody preparation method based on the sequence information of the marker gene.

### ===Diagnostic Kit Including the Diagnostic Marker===

A diagnostic kit which can be used for a simple diagnosis of migraine can be provided by using detection reagents of the diagnostic marker according to the present invention. For example, when the gene-related substance serving as the diagnostic marker is an mRNA or a cDNA, the kit can contain primers to be used for quantitative PCR as well as reagents required for PCR reaction. Alternatively, the kit may contain cDNA or cRNA, or their part for the gene-related substance as a probe for use in the Northern blotting method. Further, when the gene-related substance is a protein or a polypeptide, the kit can contain reagents etc. for detection of specific primary antibodies, secondary antibodies, and enzymes bound to the secondary antibodies to be used for immunochemical assays.

### ===Screening Method Based on Drug Efficacy===

The efficacy of a therapeutic agent for migraine depends on the patient who takes it. By using the diagnostic marker according to the present invention, it is possible to screen in vitro for a therapeutic agent effective for a particular patient with migraine and thus to administer to the patient the therapeutic agent which has been selected in advance based on its efficacy.

First, when a plurality of therapeutic agents for migraine is under consideration, they are added to the lymphocytes or lymphocyte-derived immortalized cells obtained from a patient. Then, before and after this addition, the expression level of the diagnostic marker for migraine is measured. As a result, when the expression level of a certain candidate substance decreased after its addition compared with that before the addition, the therapeutic agent for migraine can be judged to have efficacy on the patient. Thus, by performing the measurement for each of a plurality of therapeutic agents for migraine and comparing the degrees of decrease in each expression level, the efficacy of each therapeutic agent for migraine can be quantitatively compared, thereby making it possible to select a therapeutic agent with the highest therapeutic effect on the patient to be treated.

Based on the standard immunochemical assay or nucleic acid assay as described above, the screening method based on the drug efficacy according to the present is characterized in that it enables treatment of a large quantity of samples in a simple manner. In addition, since cells of the person who takes the therapeutic agent for migraine are used, this screening method enables accurate, quantitative evaluation as an order-made treatment.

### ===Method for Screening a Novel Therapeutic Agent for Migraine===

A novel therapeutic agent for migraine can be screened for using the same method as described above. That is, a compound serving as a candidate for a therapeutic agent for migraine, instead of the existing therapeutic agent for migraine, is used such that it is added to lymphocytes or lymphocyte-derived immortalized cells obtained from a plurality of patients with migraine. If the expression level of the diagnostic marker decreased even in one patient, the compound can be a potential novel therapeutic agent for migraine. To confirm the possibility, it is possible to test the compound for its efficacy as a therapeutic agent for migraine by administering it directly to the patient from whom the lymphocytes exhibiting a decreased expression level of the marker are derived. If expression level of the diagnostic marker more decreased in more patients by a candidate compound, the possibility that it is a generally effective therapeutic agent for migraine is higher.

### Example

Hereinafter, the embodiments of the present invention will be explained in more detail with reference to Examples and drawings.

### ===Isolation and Immortalization of Lymphocytes===

Peripheral bloods were harvested from 5 patients having an aura of migraine, as diagnosed according to the International Classification of Headache Disorders 2nd Edition (ICHD-II) of the International Headache Society as well as from 5 healthy subjects constituting a control group who are matched with the 5 patients in terms of age and sex. From the migraine subjects, their bloods were drawn during an interval between migraine attacks. Their lymphocytes were isolated by centrifugation from the peripheral bloods collected from each group.

The lymphocytes of each group were infected by adding 1 mL of EB virus supernatant (the culture supernatant of B958 cells, EB virus-producing cells) to the lymphocytes at 2×10⁶ cells and 2 μL of 100-fold diluted cyclosporin solution (original solution of cyclosporin (Sandimmun 1A (5 ml) 250 mg) to the lymphocytes at 2×10⁶ cells, followed by incubation in RPMI(+) medium for about four days. Then, cell lines established by continuous passaging in the same medium were used as the lymphocyte-derived immortalized cells in the present invention. These immortalized cells were used for the following experiments under the same conditions of culture condition, number of passages, etc.

### ===Microarrays and Real-Time PCR===

The cultured immortalized lymphocytes were recovered. Total RNAs were extracted and cRNAs using the total RNAs as templates were prepared with a CodeLink Expression assay reagent kit (Amersham Bioscience K.K.). The cRNAs were labeled with Cy5-streptavidin and analysis of mRNA expression level was performed on the RNA arrays included in the aforementioned kit, on which about 20,000 known human genes are spotted as probes. The intensity of the fluorescence signal emitted from Cy5 was measured with a GenePix 4000B DNA microarray scanner (Olympus). Genes in the migraine group whose expression level is more than two times compared with the healthy group were identified. As a result, they were roughly classified into cytoskeletal genes and calcium-related genes. α-fodrin (also referred to as spectrin), a membrane-lining protein and HPCAL1 (hippocalcin-like 1), a calcium-binding protein present in the brain, were obtained as one of the cytoskeletal genes and one of the calcium-related genes, respectively.

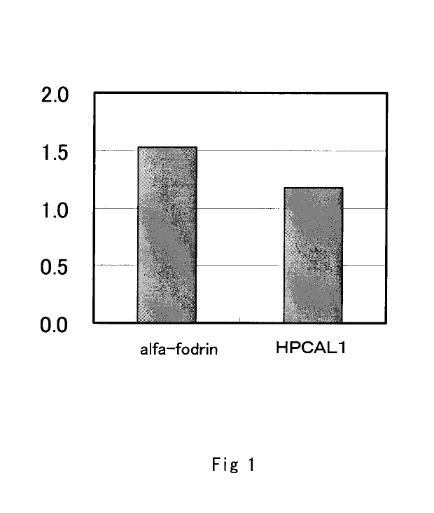

Thus, the gene expression levels of α-fodrin and HPCAL1 were examined by performing quantitative real-time PCR using immortalized lymphocytes obtained from the migraine and healthy subjects as samples. The TaqMan gene expression assay kit from Applied Biosystems, a probe included in the kit, and primers (α-fodrin: catalog No. Hs00162203_m1, HPCAL1: catalog No. Hs00365962_m1) were used to carry out the PCR. The expression level of each gene was relatively quantified by the comparative Ct (threshold cycle) method based on the amplification curve obtained from the PCR. As a result, both genes exhibited a higher expression level in the migraine group than in the healthy group (FIG. 1). This result indicates that both genes can serve as markers for migraine.

### ===Evaluation of Efficacy===

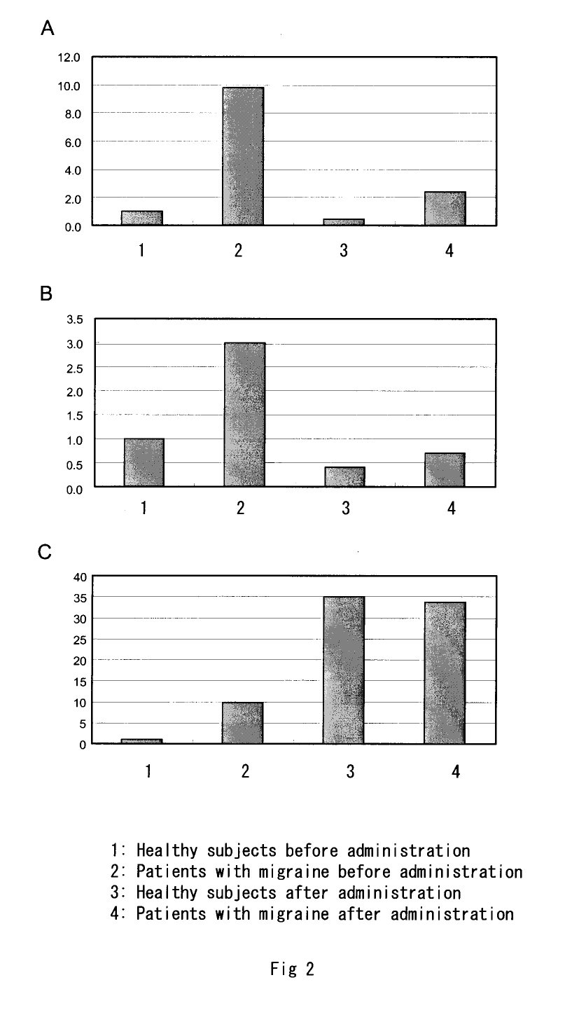

Immortalized cells derived from lymphocytes of the migraine group and the healthy group were subjected to mRNA expression analysis in the same manner as described above. In this analysis, sumatriptan, a tiptan therapeutic agent for migraine, or aspirin, a commonly used anti-inflammatory painkiller, was administered to the immortalized cells in culture. By using part of the immortalized cells before and after the administration of each drug, the expression level of α-fodrin gene was analyzed. As a result, both sumatriptan and aspirin reduced the expression level of α-fodrin (FIG. 2A, B). The decrease was larger in the cells derived from the migraine subjects than in those derived from the healthy subjects. In contrast, when Gaster (famotidine), a stomach drug, was administered to the immortalized cells in the same manner, the drug did not reduce but, on the contrary, increased the expression level of α-fodrin in the cells derived from migraine subjects as well as those derived from healthy subjects (FIG. 2C).

Thus, it has been shown that, by using the assay method according to the present invention, the efficacy of a therapeutic agent for migraine can be evaluated. This assay method can be applied to the screening method based on drug efficacy and the screening method for a novel therapeutic agent for migraine.

### ===Use of the Diagnostic Marker in Animal Model===

It is said that, in the pathological condition of migraine, the phenomenon of cortical spreading depression (CSD) is highly likely to have occurred in the cerebral cortex. Thus, CSD was experimentally induced by the following method using mice and the expression of α-fodrin in the cerebral cortex was examined.

Two bilateral pairs of holes (1 cm) were drilled through the skull of male C57/Black6 mice. The dura mater was removed and electrodes were placed on the brain surface. CSD was induced by stimulation by dribbling 0.3 M KCI with a micro pipette through the right hole, which was 5 mm away from the electrodes. Then, total RNAs were extracted from the stimulated and non-stimulated cerebral hemispheres and cDNAs were prepared with reverse transcriptase. The expression level of α-fodrin was measured by the real-time PCR method using a TaqMan probe and the ABI PRISM 7700 Sequence Detection System (Applied Biosystems).

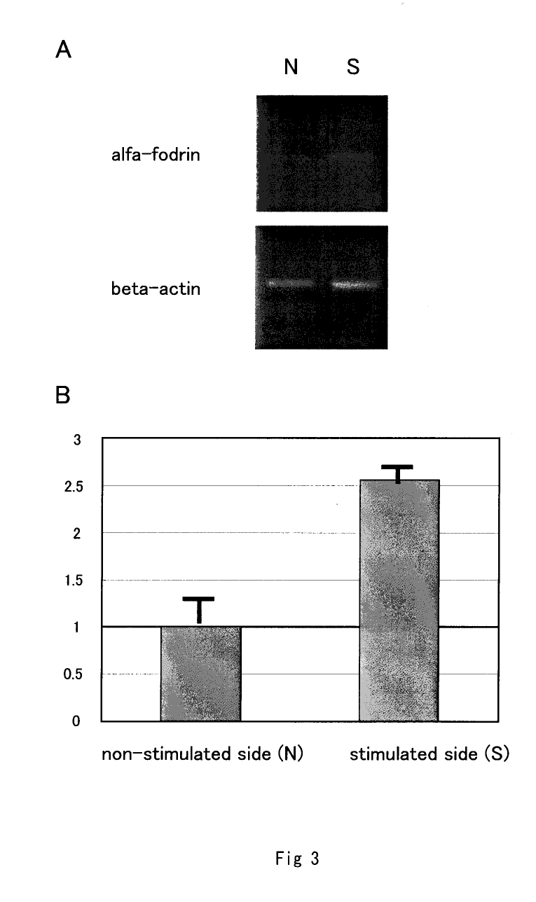

As a result, in the cerebrum of the mice, the expression of α-fodrin in the cerebral hemisphere in which CSD had occurred was higher than that in the cerebral hemisphere in which CSD had not occurred (FIG. 3A, B). In conclusion, it was clarified that α-fodrin can be used as a diagnostic marker of migraine.

### INDUSTRIAL APPLICABILITY

Diagnostic markers that enable diagnosis of migraine in a simple and convenient manner and methods for using the same can be provided by the present invention.

1. (canceled)

2. (canceled)

3. A diagnostic kit for migraine comprising a reagent for detecting expression of an α-fodrin gene or an HPCAL1 gene.

4. An assay method comprising the step of examining an expression level of the α-fodrin gene or the HPCAL1 gene in a lymphocyte isolated from a vertebrate.

5. The assay method of claim 4, wherein the isolated lymphocyte has been immortalized.

6. (canceled)

7. A method for screening for a therapeutic agent for migraine, the agent being effective for a patient with migraine, comprising the steps of:
(a) administering therapeutic agents for migraine to a lymphocyte isolated from the patient with migraine, and
(b) examining expression level of the α-fodrin gene or the HPCAL1 gene in the isolated lymphocyte before and after the administration of the agents, wherein
a therapeutic agent that reduces the expression level is selected.

8. A method for screening a novel therapeutic agent for migraine comprising the steps of:
(a) administering a substance serving as a candidate for a therapeutic agent for migraine to a lymphocyte isolated from the patient with migraine,
(b) examining expression level of the α-fodrin gene or the HPCAL1 gene in the isolated lymphocyte before and after the administration, wherein
a candidate substance that reduces the expression level is selected.

9. The screening method of claim 8 wherein the expression level of the α-fodrin gene or the HPCAL1 gene is examined by measuring the amount of mRNA, cDNA, or protein derived from the gene.

10. The screening method of claim 8, wherein the isolated lymphocyte has been immortalized.

11. (canceled)

12. A method for diagnosing migraine comprising examining expression level of the α-fodrin gene or the HPCAL1 gene in a lymphocyte isolated from a subject.

13. A method for diagnosing migraine, comprising the steps of:
(a) administering a therapeutic agent for migraine to a lymphocyte isolated from a subject,
(b) examining an expression level of the α-fodrin gene or the HPCAL1 gene in the isolated lymphocyte before and after the administration of the agent, and
(c) diagnosing the subject having the expression level reduced by the administration of the agent as being afflicted with migraine.

14. The diagnosis method of claim 12, wherein the expression level of the α-fodrin gene or the HPCAL1 gene is examined by measuring the amount of mRNA, cDNA, or protein derived from the gene.

15. (canceled)

16. (canceled)

17. The diagnosis method of claim 13, wherein the expression level of the α-fodrin gene or the HPCAL1 gene is examined by measuring the amount of mRNA, cDNA, or protein derived from the gene.

18. The screening method of claim 7 wherein the expression level of the α-fodrin gene or the HPCAL1 gene is examined by measuring the amount of mRNA, cDNA, or protein derived from the gene.
